(19) United States
(12) Patent Application Publication
(10) Pub. No.: US 2017/0311941 A1
(43) Pub. Date: Nov. 2, 2017

(54) STERNAL RETRACTOR WITH RELEASABLE ARMS HAVING SNAP LOCKS

(71) Applicant: Scanlan International, Inc., St. Paul, MN (US)

(72) Inventors: Jean E. Daavettila, St. Paul, MN (US); Scott E. Jahns, Hudson, WI (US)

(21) Appl. No.: 15/581,680

(22) Filed: Apr. 28, 2017

Related U.S. Application Data
(60) Provisional application No. 62/328,826, filed on Apr. 28, 2016.

(51) Int. Cl. A61B 17/02; A61B 90/00; A61B 17/00

(52) U.S. Cl. CPC A61B 17/0206; A61B 2017/0092; A61B 2090/3966; A61B 2017/00473

(57) ABSTRACT

A surgical retractor comprises a rack, a pair of adapters on the rack, and a pair of arms with blades that are releasably connectable to the adapters. Each arm includes a pair of snap arms having top and bottom sides, a head on a distal end, and an undercut in both of the top and bottom sides at a proximal portion of the head. Each adapter includes a channel having undercut catches, and is configured to receive the retractor arm at an inserted position. The undercut catches extend into the channel and engage the snap arm undercuts when the retractor arm is at the inserted position. Lead-ins in the channel are configured to engage the snap arm heads and deflect the heads around the undercut catches as the retractor arm is inserted into the channel and moved toward the inserted position. The retractor arms and adaptors can be formed from radiodense and/or radiolucent materials.

### CROSS REFERENCE TO RELATED APPLICATION

This application claims the benefit of U.S. Provisional Application Ser. No. 62/328,826, filed Apr. 28, 2016 and entitled Sternal Retractor with Releasable Arms Having Snap Locks, which is incorporated herein by reference in its entirety and for all purposes.

### FIELD OF THE INVENTION

The invention relates generally to surgical instruments. In particular, embodiments of the invention include sternal and other retractors.

### BACKGROUND

Surgical devices such as sternal and other retractors are generally known and commercially available. Devices of these types are often made from metal such as stainless steel or titanium. Materials of these types can block the transmission of x-rays and thereby interfere with or prevent the imaging of anatomical features near the devices. Materials that inhibit the passage of imaging electromagnetic radiation are sometimes called radiodense.

During complex surgical procedures, surgeons often perform radiographic imaging to determine the anatomical and physiological state of the patient. Removing a metal retractor to allow for imaging during such a surgical procedure may take time and disturb the procedure, thereby increasing the risk of complications. Materials that allow the transmission of imaging electromagnetic radiation are sometimes called radiolucent.

There remains a continuing need for improved surgical retractors. In particular, there is a need for retractors that are radiolucent at the surgical site. Any such retractors should be highly functional and capable of being efficiently used.

### SUMMARY

A retractor in accordance with embodiments of the invention comprises a rack, an adapter on the rack, and an arm releasably connectable to the adapter. A first connector structure is on one of the adapter and the arm. The first connector structure includes an end portion comprising at least one snap arm having top and bottom sides, a head on a distal end, and an undercut in one or both of the top and bottom sides of each snap arm. The undercut is optionally at a proximal portion of the head. A second connector structure is on the other of the adapter and the arm. The second connector structure includes a channel configured to receive the end portion of the first connector structure, including each snap arm and head, at an inserted position. An undercut catch associated with each undercut extends into the channel, and engages each undercut when the end portion of the first connector structure is at the inserted position. Embodiments include a lead-in in the channel associated with each snap arm. The lead-in is configured to engage the head and deflect the head around the undercut catch as the end portion of the first connector structure is inserted into the channel and moved toward the inserted position.

### BRIEF DESCRIPTION OF THE DRAWINGS

FIG. 1 is an isometric view of a sternal tissue/bone retractor in accordance with embodiments of the invention.

FIG. 2 is a plan view of the retractor shown in FIG. 1.

FIG. 3 is an isometric view of the stationary adapter shown in FIG. 1, illustrating a first side and first end of the adapter.

FIG. 4 is an isometric view of the stationary adapter shown in FIG. 3, illustrating the first side and a second end opposite the end shown in FIG. 3.

FIG. 5 is side view of the stationary adapter shown in FIG. 3.

FIG. 6 is a sectional view of the adapter shown in FIG. 5, taken along line 6-6 in FIG. 5.

FIG. 7 is an end view of the stationary adapter shown in FIGS. 3 and 4, illustrating the end shown in FIG. 4.

FIG. 8 is a sectional view of the adapter shown in FIGS. 3 and 4, taken along line 8-8 in FIG. 7.

FIG. 9 is an isometric view of the moving adapter shown in FIG. 1, illustrating a first side and first end of the adapter.

FIG. 10 is an isometric view of the moving adapter shown in FIG. 9, illustrating the first side and a second end opposite the end shown in FIG. 9.

FIG. 11 is a side view of the adapter shown in FIG. 10, illustrating the first side shown in FIG. 10.

FIG. 12 is an end view of the adapter shown in FIG. 9, illustrating the first end shown in FIG. 9.

FIG. 13 is an end view of the adapter shown in FIG. 10, illustrating the second end shown in FIG. 10.

FIG. 14 is a sectional view of the adapter shown in FIG. 13, taken along line 14-14 in FIG. 13.

FIG. 15 is an isometric view of the moving or left-side arm shown in FIG. 1, illustrating a first side of the arm and a first side of a blade on the arm.

FIG. 16 is a bottom side view of the arm shown in FIG. 15, illustrating the first side shown in FIG. 15.

FIG. 17 is a top side view of the arm shown in FIG. 15, illustrating a second side opposite the side shown in FIG. 15.

FIG. 18 is an edge or side view of the arm shown in FIG. 15, illustrating a second side of the blade opposite the side shown in FIG. 15.

FIG. 19 is a detailed edge view of the snap arm of the arm shown in FIG. 18.

FIG. 20 is a sectional view of the snap arms of the arm shown in FIG. 16, taken along line 20-20 in FIG. 16.

FIGS. 21A-21C are detailed cross sectional views of the moving adapter and arm, illustrating the end of the arm at a sequence of positions during insertion into the adapter.

FIG. 22 is a detailed edge or side view of the retractor shown in FIG. 1, illustrating the stationary arm in the locked position in the stationary adapter.

FIG. 23 is a detailed end view of the retractor shown in FIG. 1, illustrating the movable arm in the locked position in the movable adapter.

### DETAILED DESCRIPTION

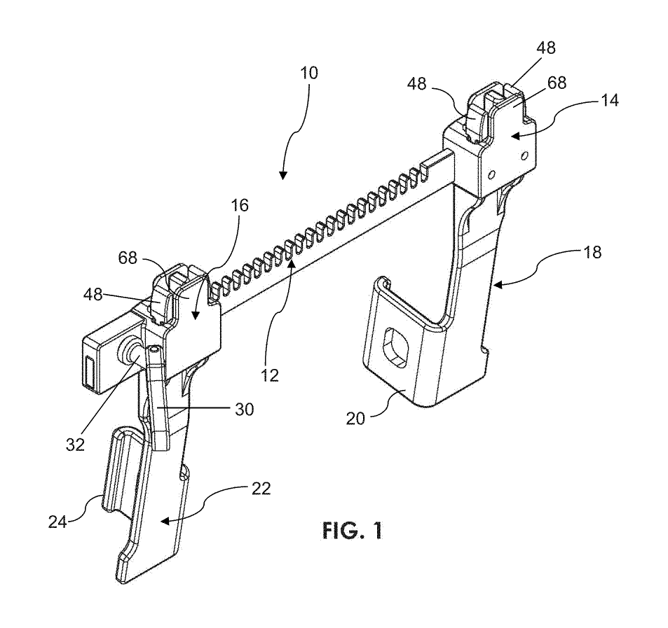

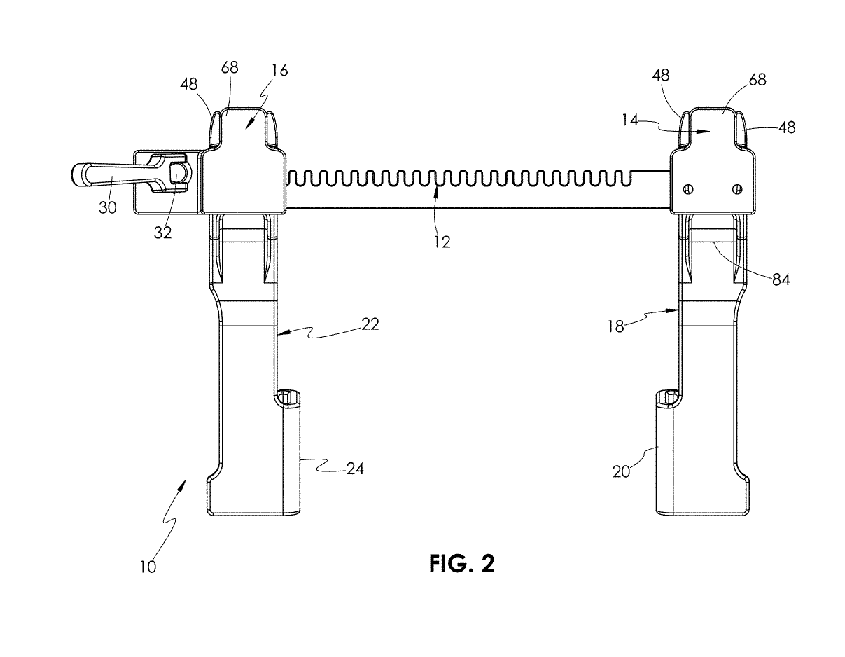

A sternal retractor 10 in accordance with embodiments of the invention can be described generally with reference to FIGS. 1 and 2. As shown, the retractor 10 includes a rack 12, stationary adapter block or adapter 14, movable adapter block or adapter 16, first (e.g., right) arm 18 having a blade 20, second (e.g., left) arm 22 having a blade 24, crank 30 and pinion assembly 32. Stationary adapter 14 is generally fixedly mounted to one end of the rack 12, and the movable adapter 16 is movably attached to the rack between the opposite end and the stationary adapter. As described in greater detail below, during use of the retractor 10 the arms 18 and 22 (which can be metal such as titanium or high density polymers) can be selected from sets of differently sized and packaged sterilized arms, removed from the packaging, and inserted into the adapters 14 and 16, respectively. Snap lock connector structures on the adapters 14 and 16 and arms 18 and 22 securably engage the arms to the adapters. During surgical procedures, the crank 30 of retractor 10 can be actuated to rotate the pinion assembly 32 and thereby move the movable adapter 16 and arm 22 attached thereto with respect to the stationary adapter 14 and arm 18. After the use of the retractor 10, the snap lock connector structures can be actuated to release the arms 18 and 22, and the arms can be removed from the adapters 14 and 16, respectively. The previously used arms 18 and 22 can be disposed of or sterilized and repackaged for another use.

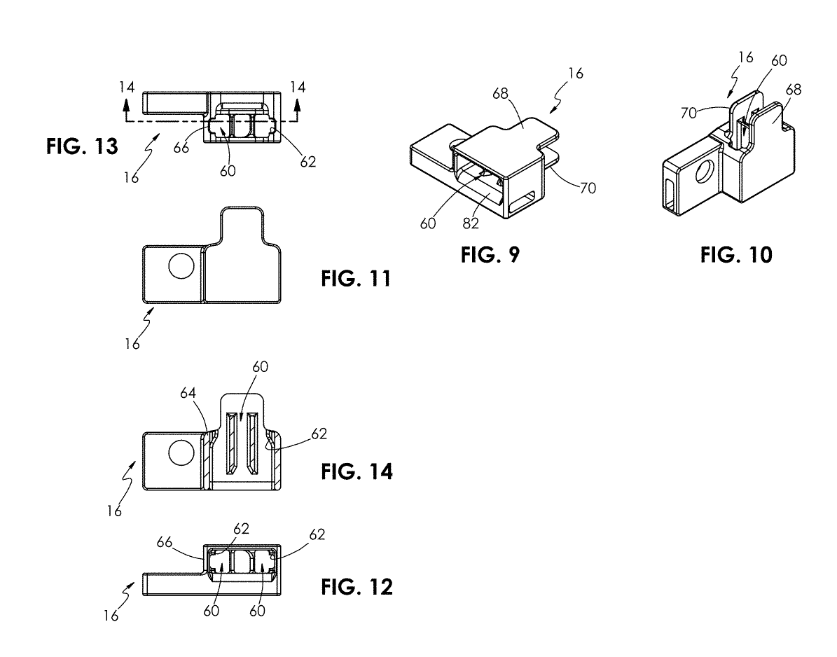

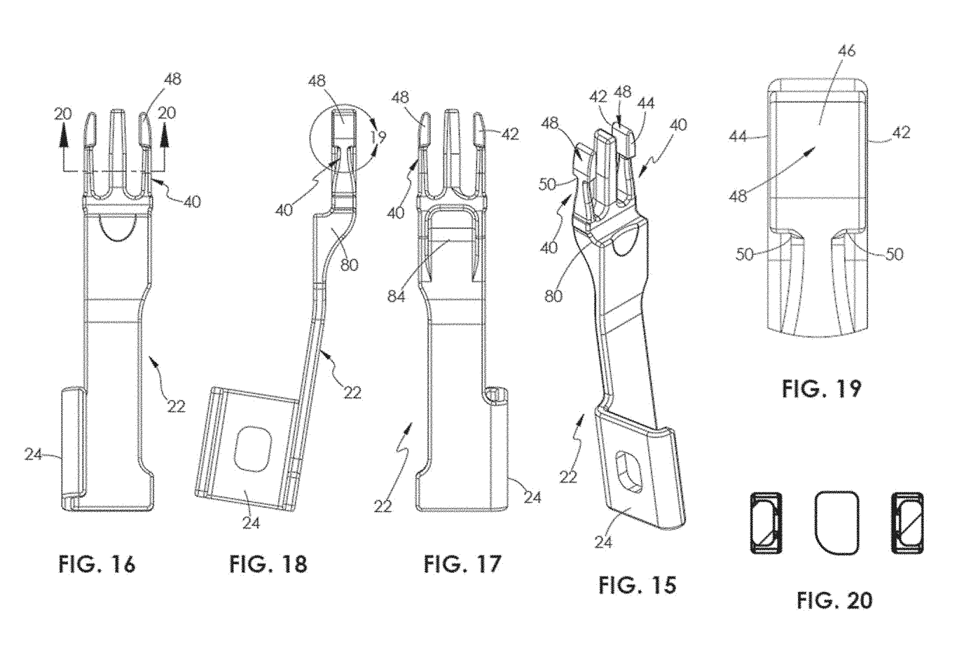

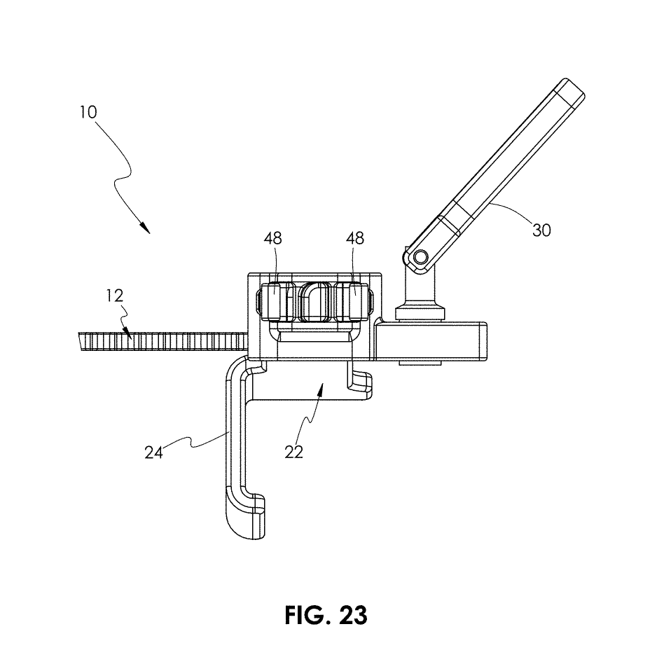

Other features of retractors 10 in accordance with embodiments of the invention can be described with reference to FIGS. 1-23. The retractor has adapters 14 and/or 16 and arms 18 and/or 22 that are configured to be releasably connected. A first connector structure is on one of the adapter 14 and/or 16 and the arm 18 and/or 22. In the illustrated embodiment (e.g., FIGS. 15-20) the first connector structure includes an end portion on the arms 18 and/or 22 having at least one snap arm 40 (two are shown). Snap arms 40 have top and bottom (i.e., first and second) sides 42 and 44, respectively, a lateral side 46, and a head 48 on a distal end of the snap arm. An undercut 50 is in one or both of the top side 42 and bottom side 44 of each snap arm 40. In the illustrated embodiment, each undercut 50 is at a proximal portion of the head 48, but can be at other locations in other embodiments (not shown).

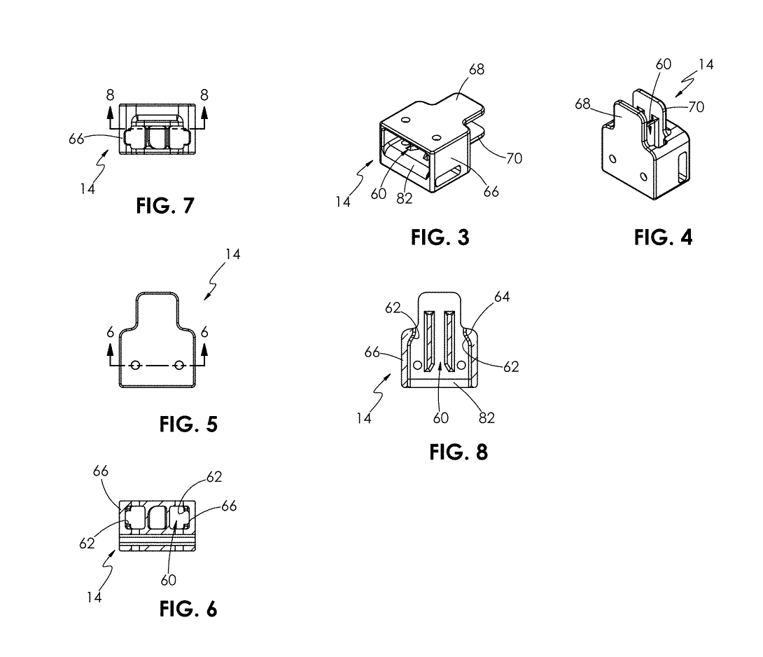

A second connector structure is on the other of the adapter 14 and/or 16 and the arm 18 and/or 22. In the illustrated embodiment (e.g., FIGS. 3-14), the second connector structure is on the adapters 14 and/or 16 and includes a channel 60 configured to receive the end portion of the first connector structure, including each snap arm 40 and head 48, at an inserted position. The second connector structure also includes an undercut catch 62 associated with each snap arm undercut 50. The undercut catches 62 extend into the channel 60 to engage each snap arm undercut 50 when the end portion of the first connector structure is at the inserted position. In the illustrated embodiments the second connector structure also includes a lead-in 64 in the channel 60 associated with each snap arm 40. Each lead-in 64 is configured to engage the head 48 and deflect the head around the undercut catch(s) 62 as the end portion of the first connector structure is inserted into the channel 60 and moved toward the inserted position.

In the illustrated embodiments, the first connector structure includes an undercut 50 in the top side 42 and bottom side 44 of each snap arm 40. The second connector structure includes first and second undercut catches 62. The first undercut catch 62 engages the undercut 50 in the top side 42 of the snap arm 40, and the second undercut catch engages the undercut in the bottom side 44 of the snap arm.

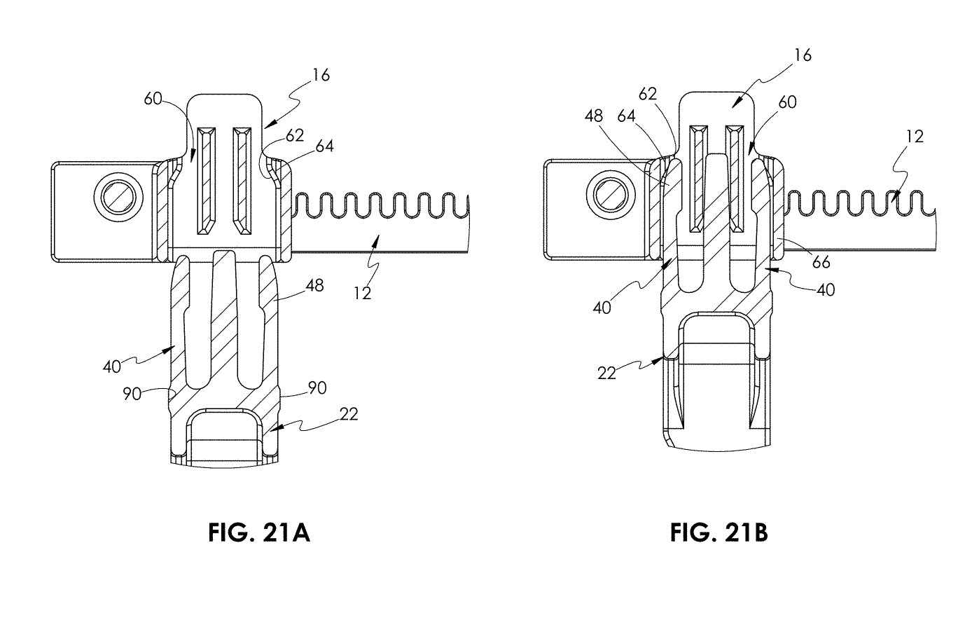

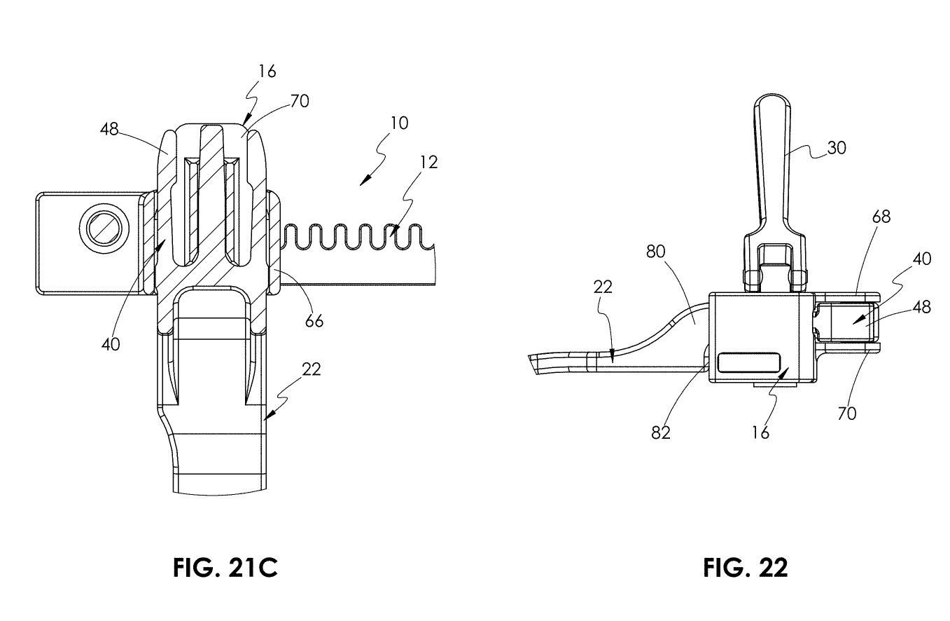

The first connector structure includes two snap arms 40 at laterally spaced locations on the end portions of the arms 18 and 22 in the illustrated embodiments. The second connector structure in the adapters 14 and 16 in the illustrated embodiments includes a channel 60 having two laterally spaced side walls 66. As shown (e.g., FIG. 21B), the lead-ins 64 deflect the snap arm heads 48 inwardly into the channel 60 (i.e., toward each other) as the end portion of the first connector structure is inserted into the channel. The lead-in 64 can deflect the snap arm 40 around the undercut catch 62 as the end portion of the first connector structure is inserted into the channel 60 and moved toward the inserted position.

In embodiments (e.g., FIGS. 2, 21C), the channel 60 of the second connector structure includes a top surface or wall portion 68 and/or a bottom surface or wall portion 70 extending over at least portions of the top side 42 and/or at least portions of the bottom side 44 of each snap arm head 48 when the first connector structure is at the inserted position in the channel. The top surface or wall portion 68 and/or the bottom surface or wall portion 70 extends at least to a distal end of the snap arm head 48 when the first connector structure is at the inserted position in the channel in embodiments. As shown (e.g., FIGS. 3, 4, 8, 9) the second connector structure includes a tubular structure defining the channel 60 in the adapters 14 and 16.

The end portion of the first connector structure extends from a shoulder 80 on the arms 18 and 22 in the illustrated embodiment (e.g., FIGS. 17, 21). The second connector structure includes an edge 82 in the adapters 14 and/or 16 that is engaged by the shoulder 80 of the first connector structure when the first connector structure is in the inserted position. A gusset 84 is located on a portion of the first connector structure (e.g., arms 18 and/or 22) adjacent to the shoulder 80 in the illustrated embodiments.

In embodiments, the first connector structure is on an end of a retractor arm such as 18 and/or 22, and optionally on an end of a sternal retractor arm, a self-retaining retractor arm, or a spinal retractor arm (e.g., a so-called Adson retractor). The retractor arms 18 and/or 22 can include (1) only radiolucent material, (2) only radiodense material, or (3) both of radiolucent and radiodense material. In embodiments, the second connector structure includes (1) only radiolucent material, (2) only radiodense material, or (3) both of radiolucent and radiodense material. In yet other embodiments, the first connector structure includes (1) only radiolucent material, (2) only radiodense material, or (3) both of radiolucent and radiodense material, and the second connector structure includes (1) only radiolucent material, (2) only radiodense material, or (3) both of radiolucent and radiodense material. Embodiments of the invention include packaged sterile retractor arms such as 18 and/or 22 having the first or second connector structures of the types described above, and optionally different types (e.g., left and right) and sizes of such arms. Similarly, embodiments include packaged sterile retractor adapters such as 14 and/or 16 having the first or second connector structures of the types described above, and optionally different types (e.g., left and right) of such adapters.

Embodiments of the invention offer important advantages. They can, for example, allow x-ray and other imaging of target tissues. They can incorporate the use of metals outside of the target, thereby providing strength, reuse and other high-quality functionality. The ability to efficiently attach and detach various sizes and configurations of arms and blades offers efficiencies by saving the surgeon time and providing optimal fitting to the patient and procedural needs. For example, the device can be configured for pediatric, adolescent and adult patients. The retractors can accommodate both radiolucent (e.g., polymer) and radiodense (e.g., titanium, stainless steel) arms and blades.

Snap buckle designs are used for binding applications such as on sleeping bags, back packs, etc. In these applications the force that is applied to the snap buckle places it in tension. The snap structures of the embodiments described above primarily are subjected to a torsional force. The force is generated by the spreading of the tissue and bone during operation of the retractors 10, which transmits a force to the arms 18 and/or 22 and blades 20 and/or 24. This force in turn produces a torque load between the snap arms 40 and the adapters 14 and 16.

To accommodate the torque forces, the snap structures incorporate certain design details in embodiments. The shoulder 80 and edge 82 function as a torque step for the stationary and moveable arms 18 and/or 22. The step feature provides two functions. A first is that it allows the bottom of the arm 18 and/or 22 to stay in a planer arrangement with the bottom of the rack 12. This is desirable for the surgeon so that the instrument sits flat on the patient's chest while minimizing the length of the arms 18 and/or 22. If the attachment mechanism was put into the plane of patient's chest it may cause the length of the arm/rack system to grow longer. A goal is to allow x-ray imaging to maximize the radiolucent zone, so the metal components are preferably outside the x-ray viewing area. A second is that the torque step transmits a portion of the forces of arms 18 and/or 22 to the mating surfaces of the adapter blocks 14 and/or 16.

When the arms 18 and/or 22 are inserted into the adapters 14 and/or 16, the lead-ins 64 provide a lead-in for ease of insertion by the medical staff. Because of these lead-ins 64, the head 48 end of the arms 18 and/or 22 may be moveable if not for the features of the lock-up pads 90 (FIG. 20) and the torque steps provided by the shoulder 80 and edge 82. These features have a near line to line interface with the mating surfaces on the adapter 14 and/or 16, which contributes to the arms 18 and/or 22 being laterally steady when fully seated.

There are four undercut catches 62 located on each adapter 14 and 16 of the illustrated embodiment. The snap arms 40 and undercuts 50 on each of the two arms 40 for each retractor arm 18 and 22 are deflected inward from their neutral positions during insertion of the arms into the adapter blocks 14 and 16 by the lateral side 46 of the arms coming into contact with the lead-ins 64 located in four positions on each adapter block. When the arms 18 and /or 22 are fully inserted the elastic memory (Kinetic Energy) or resilient nature of the deflected arms causes the heads 48 and thus the undercuts 50 to move outwardly toward their neutral positions when they have cleared the undercut catches 62. Upon the arms 18 and/or 22 returning to their natural position, the arms are in the locked position, preventing them from being withdrawn from the adapters 14 and/or 16. When tension is applied to withdraw the locked arms 18 and/or 22, the undercuts 50 impinge upon the undercut catches 62 causing a mechanical obstruction blocking the withdrawal of the arms.

To withdrawal the arms 18 and/or 22 a person can apply an inward force, such as with their fingers, to the heads 48 on the ends of the arms 40 while applying a withdrawing tension on the retractor arms 18 and/or 22. A design feature in embodiments is the incorporation of geometry to prevent accidental compression of the snap arms 40 that could lead to disengagement of the arms. The top surfaces 42 and bottom surfaces 44 of the heads 48 are maintained below the top surfaces 68 and bottom surfaces 70 of the adapter blocks 14 and/or 16. The heads 48 can be designed not to protrude beyond the rear edge of the top surfaces 42 and bottom surfaces 44. The portion of the adapter block 14 and/or 16 that extends along the rack 12 is designed to the specified width to shield the heads 48 from accidental contact and disengagement in embodiments.

Although the invention has been described with reference to preferred embodiments, those of skill in the art will recognize that changes can be made in form and detail without departing from the spirit and scope of the invention.

What is claimed is:

1. A retractor, comprising:
a rack;
an adapter on the rack;
an arm releasably connectable to the adapter;
a first connector structure on one of the adapter and the arm, the first connector structure including an end portion comprising:
at least one snap arm having top and bottom sides, and a head on a distal end; and
an undercut in one or both of the top and bottom sides of each snap arm, wherein each undercut is optionally at a proximal portion of the head; and
a second connector structure on the other of the adapter and the arm, the second connector structure comprising:
a channel configured to receive the end portion of the first connector structure, including each snap arm and head, at an inserted position;
an undercut catch associated with each undercut and extending into the channel, to engage each undercut when the end portion of the first connector structure is at the inserted position; and
optionally a lead-in in the channel associated with each snap arm, the lead-in configured to engage the head and deflect the head around the undercut catch as the end portion of the first connector structure is inserted into the channel and moved toward the inserted position.

2. The retractor of claim 1 wherein:
the first connector structure includes an undercut in the top and bottom sides of each snap arm; and
the second connector structure includes first and second undercut catches, wherein the first undercut catch engages the undercut in the top side of the snap arm, and the second undercut catch engages the undercut in the bottom side of the snap arm.

3. The retractor of claim 1 wherein:
the first connector structure includes two snap arms at laterally spaced locations on the end portion; and
the second connector structure includes a channel having two laterally spaced side walls; and wherein the lead-ins deflect the snap arm heads inwardly into the channel as the end portion of the first connector structure is inserted into the channel.

4. The retractor of claim 1 wherein the channel of the second connector structure includes a top wall portion and/or a bottom wall portion extending over at least portions of the top and/or at least portions of the bottom of each snap arm head when the first connector structure is at the inserted position in the channel.

5. The retractor of claim 4 wherein the top wall portion and/or the bottom wall portion extend at least to a distal end of the snap arm head when the first connector structure is at the inserted position in the channel.

6. The retractor of claim 1 wherein the second connector structure includes a tubular structure defining the channel.

7. The retractor of claim 1 wherein:
the end portion of the first connector structure extends from a shoulder; and
the second connector structure includes an edge that is engaged by the shoulder of the first connector structure when the first connector structure is in the inserted position.

8. The retractor of claim 7 and further including a gusset on a portion of the first connector structure adjacent to the shoulder.

9. The retractor of claim 1 wherein the first connector structure is on an end of the retractor arm.

10. The retractor of claim 9 wherein the retractor arm includes (1) only radiolucent material, (2) only radiodense material, or (3) both of radiolucent and radiodense material.

11. The retractor of claim 9 wherein the second connector structure is on the adaptor.

12. The retractor of claim 11 wherein the second connector structure includes (1) only radiolucent material, (2) only radiodense material, or (3) both of radiolucent and radiodense material.

13. The retractor of claim 1 wherein:
the first connector structure includes (1) only radiolucent material, (2) only radiodense material, or (3) both of radiolucent and radiodense material; and
the second connector structure includes (1) only radiolucent material, (2) only radiodense material, or (3) both of radiolucent and radiodense material.

14. A component for a surgical retractor, wherein the component includes an end portion comprising:
at least one surgical retractor component snap arm having top and bottom sides, a lateral side, and a head on a distal end; and
an undercut in one or both of the top and bottom sides of each snap arm, wherein each undercut is optionally at a proximal portion of the head.

15. The component of claim 14 wherein the snap arm includes an undercut in the top and bottom sides of each snap arm.

16. The component of claim 14 comprising two laterally spaced snap arms.

17. The component of claim 16, wherein the component comprises a retractor arm including a blade.

18. A packaged and sterile retractor arm in accordance with claim 17.

19. A first component for a surgical retractor, wherein the first component is configured to receive an end portion of a second component having a snap arm having top and bottom sides, a head, and an undercut in one or both of the top and bottom sides of the snap arm, the first component comprising:
a surgical retractor component channel configured to receive the end portion of the first connector structure, including the snap arm and head, at an inserted position; and
an undercut catch associated with each snap arm undercut and extending into the channel, to engage each undercut when the snap arm is at the inserted position.

20. The first component of claim 19, wherein the first component further comprises a lead-in in the channel associated with the snap arm, the lead-in configured to engage the snap arm head and deflect the head around the undercut catch as the snap arm is inserted into the channel and moved toward the inserted position.

21. The first component of claim 20, wherein the first component comprises a retractor adapter configured to be mounted to a rack.

22. A packaged and sterile retractor adapter in accordance with claim 21.
